(19) United States
(12) Patent Application Publication
(10) Pub. No.: US 2015/0050245 A1
(43) Pub. Date: Feb. 19, 2015

(54) METHODS AND DELIVERY SYSTEM FOR BENEFICIAL ADDITIVE AND SUBTRACTIVE BIOLOGICAL AGENTS TO PROMOTE BALANCED MAMMALIAN GASTROINTESTINAL FLORA

(71) Applicant: TNTGamble, Inc., Redmond, WA (US)

(72) Inventors: Richard E. Herman, Redmond, WA (US); Timothy Gamble, Seattle, WA (US)

(21) Appl. No.: 14/459,911

(22) Filed: Aug. 14, 2014

Related U.S. Application Data
(60) Provisional application No. 61/866,025, filed on Aug. 14, 2013.

(51) Int. Cl. A61K 35/74; A61K 9/20; A61K 9/00; A61K 35/76

(52) U.S. Cl. CPC A61K 35/744; A61K 35/76; A61K 9/2054; A61K 9/2063; A61K 9/0053; A61K 9/2009; A61K 9/205
USPC 424/93.3

(57) ABSTRACT

This invention relates to controlled release formulations of probiotic bacteria and bacteriophages, including combined formulations of probiotic bacteria and bacteriophages. The formulations contain a hydrophilic agent, an electrolytic agent and a polysaccharide and may be in tablet form for oral delivery to the intestinal system. In one preferred aspect, the formulations of the present invention effect the simultaneous introduction or addition of probiotic bacteria and bacteriophages to cause the subtraction or removal of undesirable bacteria.

### REFERENCE TO RELATED APPLICATION

This application claims the benefit of U.S. Provisional Application No. 61/866,025, titled “Probiotic Combination of Bacteria and Bacteriophages” filed Aug. 14, 2013. The contents of this application are incorporated here in their entirety by this reference.

### BACKGROUND

The gastrointestinal tract (GI) is an important and complex organ with digestive, immunological, and neurological functions. The GI typically contains a complex milieu of microorganisms that includes various bacteria as well as bacteriophages.

Classic or conventional probiotics are designed to supplement, add, or deliver live bacteria or other components to the intestine of a host in order to provide a beneficial effect, but this addresses only some of the biological agents, including various microorganisms, with potential to improve intestinal health via inclusion. One of the suggested advantages to adding beneficial bacteria in the GI is that they can displace detrimental (herein the term “detrimental” is synonymous with non-beneficial, disadvantageous, and unwanted) bacteria thereby controlling their population through competitive exclusion. Bacteriophages, however, have been largely ignored in the classic or conventional probiotics context. Nonetheless, the inventors have determined that bacteriophages can have a distinct subtractive role in microbial population dynamics that can also be used probiotically to control detrimental or undesirable bacterial populations (regardless of whether the target bacteria are pathogenic) by causing or promoting their lysis, subtraction, elimination, or removal.

Bacteriophages are viruses that specifically replicate within a bacterial cell thereby relying on bacterial cells as hosts for replication and can result in host cell death. The host range for each bacteriophage is specific and limited. Thus, unlike antibiotics, which are non-specific and inhibit or kill detrimental and beneficial bacteria alike, bacteriophages are very specific for the bacterial host that they target and therefore can be selected to target detrimental bacteria while leaving beneficial bacteria unaffected. Conventionally, bacteriophages have been used to specifically treat pathogenic bacterial infections. Specifically, there has been an interest in utilizing bacteriophages (due to their lytic specificity, adaptability, and replicative properties) in therapeutic roles as possible adjuncts to chemical antibiotics that are becoming less effective as pathogenic bacteria become more resistant to antibiotics.

As substances pass through the human gastrointestinal (GI) tract they are subjected to a wide range of pH values ranging from the neutral pH of the mouth, to the acidic conditions of the stomach, to the 5.0-7.5 pH range of the intestinal tract. Because the majority of biologically active agents, including but not limited to, probiotic microbes and bacteriophages, are highly pH sensitive, these changes in pH can cause significant effects upon the biological agent's stability and functional ability in vivo. For example, many proteins denature in acidic environments and, once denatured, their biological activity, if still present, significantly differs from the non-denatured state. For biological agents (BAs), such as, for example, bacteria and bacteriophages, to be functional, they must survive in the gastrointestinal tract with minimal exposure to pH fluctuations. Further, BAs are also sensitive to enzymatic degradation. For example, one barrier to the oral administration of insulin is its susceptibility to enzymatic degradation.

The oral administration of BAs without a controlled release system has a significant disadvantage of not allowing for the BAs to by-pass the low pH and enzyme-rich environment of the stomach, thereby potentially decreasing the viability and/or activity of the BAs. For those devices which employ an enteric coating mechanism to survive the gastric environment, the shortcomings may be two-fold. First, the process of coating the dosage form or its contents may result in significantly lowered viability of the BAs. Second, the downfall of merely by-passing the stomach is the explosive or immediate release delivery of the BAs upon exiting the stomach. This non-specific delivery is ineffectual and primitive in view of certain delivery needs of BAs because the bioavailability of BAs is often site dependent.

One or more BAs may be targeted either through modification of the BA itself or through the controlled release of the BAs within a desired physiologic window. One such BA that displays such site-specificity is the lactic acid bacteria, Lactobacillus acidophilus (a probiotic). L. acidophilus is one example of other probiotics, including Lactobacillus bulgaricus, Lactobacillus casei subsp. rhamnosus, Lactobacillus casei subsp. casei, Lactobacillus salivarius, Lactobacillus brevis, Lactobacillus reuteri, Lactococcus lactis subsp. lactis, Enterococcus faecium, Lactobacillus plantarum, Streptococcus thermophilus, Bifidobacterium infantis, Bifidobacterium bifidum, Bifidobacterium longum, Saccharomyces boulardii, and various modified soil organisms.

Each strain of L. acidophilus will attach at a different location of the intestinal tract, preferentially attaching within a region either slightly proximal or distal to other L. acidophilus strains. These preferential regions of attachment are of particular importance relative to employing the bacteria as delivery systems for genomic or proteomic therapy, whether directly or as carriers for other vectors containing genetic or proteomic biologicals.

Solid oral dosage forms employing controlled release have been increasingly demonstrated to be beneficial to the administration of pharmaceutical compounds, enhancing safety and consumer compliance, minimizing side effects, and providing new therapeutic benefits. Few have been applied to BAs due to high development costs, bioavailability issues, and stability of the dosage BAs within the dosage form. In the past, enteric coating technologies and other mechanisms of delayed release have been limited to features with explosive or immediate release delivery after the stomach.

Controlled release delivery systems can take many forms including polymeric matrix systems, wax matrix systems, multi-particulate systems, and combinations thereof. The most commonly used delivery systems can be broadly classified as diffusion, reservoir, pore forming wax, or coated-bead systems. Diffusion devices are composed of a drug dispensed in a polymer which diffuses from the entire physical tablet. Reservoir devices usually consist of a semi-permeable barrier which is involved in the release of the active from a core site within the tablet. Coated-bead systems employ an enteric or pH-sensitive coating of aggregated particles of the active ingredient packaged in capsule form. Pore forming wax systems incorporate the active ingredient into a wax base and rely upon the rate of diffusion to control the release of the active ingredient.

In tableted, pore forming wax matrices, the BAs and a water-soluble polymer are introduced into a wax or wax-like compound such as paraffin or guar gum, and then placed in an aqueous environment so as to allow the water-soluble polymer to dissolve out of the wax, resulting in the formation of pores. Upon contact with the gastrointestinal fluid, the pores facilitate diffusion-mediated release of the BAs. The rate of release of the BAs is dependent upon non-linear erosion.

Coated-bead systems are one of the few delivery systems available in both tablet and capsule form. The BAs are encased within a bead using one of the varieties of processes available, such as spheronization-extrusion or coating of non-pareils. The coated BAs are then further coated with an enteric coating or employed in a blend of coated-beads with differing release rates for extended release formulations. The BAs may also be blended or granulated with polymers before coating to provide an additional level of control. The coated-beads themselves may also be combined with polymers to create a hybrid diffusion or wax-based system. Coated-bead systems are complex to manufacture, requiring large numbers of excipients, use of solvents, and long manufacturing time. The use of such solvents and the manufacturing processes required to apply such solvents may expose the BAs to adverse environmental conditions and cause a loss of the viability of the BAs. This is especially concerning in the case of lyophilized BAs, where any exposure to moisture may cause significant decreases in viability.

An example of a reservoir system is the push-pull osmotic pump. These osmotically-controlled delivery systems feature a bi-layer tablet coated with a semi-permeable membrane possessing a laser-bored orifice through which the BAs are pushed as aqueous solution is absorbed into the tablet. There are a number of osmotic delivery systems on the market that work via a similar physical principle; these osmotic systems produce very replicable, linear release. Manufacturing this system is definitively non-conventional, requiring specialized equipment and additional processing steps. The inherent complexity of the design adds a corresponding complexity to the development and scale-up of any osmotic membrane product.

The diffusion tablet systems rely on hydrophilic polymer swelling for control of BAs release. Polymer systems can be sub-classified as conventional hydrogel systems and modified polymer systems. Conventional hydrogel systems rely upon the penetration of water to form a gel-like phase through which the bioactive agent is released. These systems often incorporate the BAs in a single polymer such as polyethylene oxide or hydroxypropyl methylcellulose. In the case of modified polymer systems, polymers with differing physical characteristics—such as one that is hydrophilic (e.g., HPMC), and one that is pH-dependent in its swelling characteristics, (e.g., pectin)—are combined with the BAs. When these polymers interact with dissolution media, a transition phase or interfacial front develops, forming a gradually dissociating semi-solid core surrounded by a gel periphery that allows the BAs to be increasingly released as the matrix hydrates. The movement of the dosage form through the gastrointestinal tract, through regions of increasing pH, permits further swelling and erosion of the matrix, culminating in complete release of the BAs and complete dissolution of the dosage form.

Prior art formulations do not address the delivery of both beneficial additive and subtractive BAs and do not deliver additive and subtractive BAs over an extended time period or to targeted individual regions of the GI tract. Prior art formulations do not incorporate the probiotic use of both bacteria and bacteriophages to balance GI microbial ecosystems in a singular controlled release formulation. These and other limitations and problems of the past are solved by the present invention which provides an improved formulation for the controlled release delivery of both beneficial additive and subtractive BAs.

### SUMMARY

The present invention is directed to a controlled release solid dosage form for one or more beneficial BAs including, but not limited to, probiotic bacteria and/or bacteriophages. In addition, the invention is directed to a method of controlled delivery of beneficial microorganisms, such as probiotic bacteria and/or bacteriophages, over an extended or within a specific timeframe to beneficially promote, treat, correct, modify, or supplement a healthy population balance of mammalian gastrointestinal flora. In a preferred embodiment, the present invention provides an advantageous combination of beneficial additive bacteria and removal or subtractive bacteriophages in a single controlled release dosage formulation for an enhanced or improved probiotic effect by directly adding beneficial bacteria while also subtracting or removing detrimental bacteria via bacteriophage action.

Beneficial microorganisms, for example, but not limited to, gastrointestinal flora such as lactic acid bacteria, yeast, and bacteriophages are essential constituents of metabolism and immune response. Supplementation or introduction of beneficial microorganisms, including probiotic microbes and bacteriophages, is a valid mechanism for replacement or modification of flora lost or altered due to antibiotic treatment, enhancing naturally-occurring levels of beneficial flora, promoting competitive inhibition, removing harmful bacteria, and otherwise preventing detrimental establishment of enteropathogens, treating diseases or health conditions, and altering the metabolism of ingested substances.

The present invention provides controlled release delivery systems for oral administration of one or more biological agents, including, for example, probiotic bacteria and bacteriophages. Further, beneficial microorganisms are delivered, including, for example at least one of probiotic bacteria and bacteriophages, to a targeted region within the GI.

Many benefits are attributed to addition of probiotic bacteria to the GI, including the ability to inhibit and competitively exclude pathogens and other detrimental bacteria in the gut. Bacteriophages are also ancillary and beneficial in that role; however, bacteriophage achieve their beneficial effect by effecting the direct removal or subtraction of undesirable bacteria. The inventors provide herein, for the first time, controlled release combination probiotic and bacteriophage formulations to harness the subtractive ability of bacteriophages to lyse specific bacteria in the gut to target and eliminate detrimental bacteria and, thus, to control detrimental bacterial populations while also providing probiotic bacteria. The inventors have determined that the strategic use of bacteriophages together with probiotic bacteria can assist the creation of space within the gut microbiome, via bacteriophage action, for the immediate probiotic bacteria occupation. Accordingly, the present invention provides an enhanced mechanism to inhibit and competitively exclude unwanted bacterial species, including unwanted bacterial species that might otherwise move in to space created by administered bacteriophage. The present invention also promotes occupation by the introduced beneficial bacterial species by creating space for these bacteria to occupy in the GI.

In a preferred embodiment, the invention comprises a combination of selected beneficial probiotic bacteria and bacteriophages, for use as a combined probiotic with effective additive and subtractive microbial population functionality, within a single dosage form. The bacteria are selected to provide the beneficial effects usually attributed to probiotic bacteria and the bacteriophages are selected to specifically target and reduce or eliminate populations of detrimental bacteria, regardless of whether these are considered pathogenic, within the gastrointestinal tract. For non-limiting examples, the beneficial bacteria may be selected from a number of candidates such as members of the lactic acid bacteria or of the genus Lactobacillus, or Bifidobacterium, or Streptococcus, and bacteriophages may be selected from a number of candidates that specifically target and replicate within bacteria identified as detrimental.

The selected bacteria and bacteriophages may be cultured for growth separately, concentrated and purified as necessary, and preserved (for example, by lyophilization). Then they may be combined at appropriate dosages within a formulation to provide a convenient way to deliver both types of native population altering microorganisms to the intestine in a single dosage form. The dosage form may be, but is not limited to, a liquid, a powder, a capsule, a caplet, a tablet, or a sachet and may comprise a combination of one or more independently derived BAs delivered together in a single dosage form.

Since the selected bacteriophages do not target the probiotic bacteria, any biological interaction of these components does not require separation of these BAs.

It is contemplated that the administration of bacteriophages may give rise to some negative effects in a case where a treated mammal has an overwhelming infection by target bacteria. This is because the lysis of many of the target bacteria in a short time frame can produce detrimental endolysins. For example, it is noted that, in some instances, successful bacteriophage therapy may be anticipated if the treated subject spikes a fever (potentially due to pyrogen release) after bacteriophage treatment.

In certain embodiments, however, the present invention can mitigate any such negative effects by delivering effective doses of bacteriophage and probiotic bacteria in a controlled release over a longer time frame and, in a preferred embodiment, filling population voids left by bacteriophage activity with the probiotic bacteria to simultaneously help prevent adsorption of toxic products to cell receptors via adherence to the GI epithelial cells in the intestinal lining and prevent the re-colonization by target bacteria or other enteropathogens.

The dosage form may also include components that protect or extend the viability of the BAs by buffering or by coating solid dosage forms with compounds designed to limit exposure to harsh conditions in the upper intestinal tract while allowing release in the remaining portion of the intestine. For example, components may be utilized that may provide a controlled release formulation, and may, optionally, include an enteric coating.

In a preferred embodiment, the bacteriophages can be selected for target bacteria that are considered to be detrimental bacteria. Because of the specificity of bacteriophages for their respective hosts, it will be possible to combine bacteriophages which target detrimental hosts in formulations together with probiotic bacteria. Intestinal microorganisms are involved in multiple diseases, for example, atherosclerosis, colon tumors, lupus, and arthritis. However, in most cases the key microorganisms have not been identified.

Generally, it is possible to find a bacteriophage that can be utilized against any target bacteria and most bacteria are susceptible to one or more bacteriophages. For example, members of the genus Prevotella are potential targets because of their involvement in the conversion of phosphatidylcholine to TMAO (trimethylamine-N-oxide) which is believed to have a role in diseases, such as arteriosclerosis, and rheumatoid arthritis, in the case of Prevotella copri. Recent scientific literature has reported that the GI of omnivores contains Prevotella bacteria that convert L-carnitine, a compound that is found in relatively high amounts in red meat, to TMAO (trimethylamine N-oxide), while vegans do not. TMAO is then metabolized by the liver resulting in the formation of compounds that contribute to arterial plaque formation, which is implicated in coronary disease. These target bacteria could be considered detrimental and therefore selected for formulation of a controlled release dosage form that provides for competitive exclusion by beneficial bacteria and for bacteriophage reduction or elimination of the target bacteria. There are instances of bacteria, however, that are not infected by any known bacteriophage, e.g., Neisseria gonorrhoeae.

In another preferred embodiment, the bacteriophages can be selected to provide, supplement, or replace beneficial or non-pathogenic bacteriophages normally ingested as part of a human or animal diet or that are typically resident in a human or animal GI. Accordingly, the present invention uniquely and advantageously provides, similar to commercial formulations of probiotic bacteria taken as dietary supplements, a dosage form that provides or supplements supplies of beneficial or non-pathogenic bacteriophages that might not otherwise be ingested in sufficient quantities.

A further advantage imparted by the controlled release combination formulations of the present invention is that multiple bacteriophages against one or more target bacteria may be incorporated into the dosage form. That is, a formulation could contain more than one bacteriophage to target a single bacterial species, and/or the formulation could contain multiple bacteriophages to enable targeting more than one bacterial species. Further, such formulations may be combined with each other, and/or with other beneficial microorganisms such as probiotic bacteria, into a dosage form permitting customized use of excipients and controlled release of the beneficial microorganisms to target areas within the GI to effect simultaneous additive and subtractive resident microflora population control.

One embodiment of a controlled delivery system includes a hydrogel or modified matrix formed from an excipient of one or more hydrophilic polymers, polysaccharides, galactomannan gums, resins, polyethylene derivatives, or hydrolyzed proteins, either alone or in combination, in which is disposed Bas. In one aspect, the beneficial microorganisms include probiotic bacteria or bacteriophages, or both, and in yet another aspect, the BAs are lyophilized and may include associated lyophilized carrier proteins. Optionally, the delivery system includes one or more additional release modifying excipients (as used herein, the terms “release modifying excipients” and “release modifying agents” are used interchangeably) from the same group of hydrophilic agents for the purpose of attenuating the release of the lyophilized ingredients with pH-specific or enzyme-specific agents, and optionally, one or more physiologically acceptable electrolytic substances included for the purpose of pH control or available water-sequestration.

In another embodiment, the controlled delivery system includes a wax matrix composed of one or more inert insoluble waxes, polymers and/or fillers, alone or in combination, in which is disposed pore forming excipients and the BAs in lyophilized form and their associated lyophilized carrier proteins.

Yet another embodiment of a controlled delivery system includes a multi-particulate system in which a plurality of granules, coated beads, or coated non-pareils are distributed within the dosage form in either a simple or a modified polymer matrix or for the purposes of controlled release of BAs in lyophilized form and their associated lyophilized carrier proteins.

Another embodiment includes a process for making an extended release dosage form, such as a tablet or capsule, from a pre-blend including mixing a BA with one or more polymers, gums, resins, polyethylene derivatives, or hydrolyzed proteins for the purpose of controlled release; the optional addition of physiologically acceptable electrolytic substances for the purpose of regulating pH within the dosage form; and the optional inclusion of available water-sequestering electrolytic species for the purpose of increasing the stability of the dosage form itself.

Another embodiment of the method of making an extended release dosage form, such as a tablet or capsule, includes mixing one or more BAs with one or more pre-blends of one or more controlling excipients, fillers, desiccants, and flow agents that has been mechanically, chemically, or otherwise dried to reduce the available water present for the purpose of preventing undesirable interactions of the BAs and hydrophilic agents with any available water within the dosage form.

The system generally includes a hydrophilic agent, an electrolyte, and one or more BAs, and may optionally include fillers, release modifying agents, desiccants, and flow agents.

In one embodiment, a delivery system is disclosed including a hydrophilic or hydrophobic agent and one or more BAs.

In another embodiment, a delivery system is disclosed including a hydrophilic agent, an electrolytic agent, and one or more BAs.

In yet another embodiment, a delivery system is disclosed including a hydrophilic agent, a release modifying agent, and one or more BAs.

In yet a further embodiment, a delivery system is disclosed including a hydrophobic agent, a release-modifying agent, and one or more BAs.

In yet a further embodiment, a delivery system is disclosed including a hydrophilic agent, electrolyte, and one or more BAs.

In yet a further embodiment, a delivery system is disclosed including a hydrophobic agent, electrolyte, and one or more Bas.

In yet a further embodiment, a delivery system is disclosed including a hydrophobic agent, release-modifying agent, electrolyte, and one or more BAs.

In yet a further embodiment, a delivery system is disclosed including a hydrophilic agent, release-modifying agent, electrolyte, and one or more BAs.

The controlled release formulations for BAs have many advantages over the current art. Targeted delivery of beneficial microorganisms, such as probiotic bacteria and bacteriophages, allows for dispersion of probiotic organisms within regions of optimal attachment or effect that may be specific to a given strain or therapeutic goal. One advantage is achieving gastric bypass for the biological contents. Another advantage of the system disclosed is the maintenance of a constant pH within the dosage form surrounding the beneficial microorganisms, allowing an optimal microenvironment for reconstitution of BAs, optionally including lyophilized ingredients, to be created, thereby maximizing viability of the ingredients released into the GI tract. Another advantage of the system disclosed is the inclusion of available water-sequestering electrolytic species such that an optimal microenvironment may be maintained during storage, thereby increasing the stability of the dosage form itself. Further advantages of the system are that it requires only dry blend and direct compression steps; the system is easily transferable to sites of manufacture and relies on only conventional tableting or encapsulation equipment for production. Because this system is relatively independent of the BAs employed in formulation, targeted delivery of probiotic bacteria, bacteriophages, genetically modified bacteria or other beneficial microorganisms is also possible.

One advantage of the present system is the controlled release of the one or more BAs from the dosage form into the surrounding environment. Another advantage of the present system is the maintenance of a constant pH within the dosage form itself through the use of physiologically acceptable electrolytic substances. Yet another advantage of the present system is the controlled exposure of the BAs within the dosage form to aqueous media through controlling the hydration rate of the dosage form via polymer disentanglement. Yet another advantage of the present system is that it increases the stability of the dosage form and the viability or activity of the BAs through the inclusion of available water-sequestering electrolytic species.

Yet another advantage of the present system is its manufacturability: a dry-blend and direct compression form can be used for tablet manufacture and a dry-blend and direct fill form can be used for capsule manufacture. Most advantageous is the absence of any processes that introduces moisture (such as coating or granulation) that may decrease the in vivo viability of the BAs.

The invention will best be understood by reference to the following detailed description of the preferred embodiment. The discussion below is descriptive, illustrative and exemplary and is not to be taken as limiting the scope defined by any appended claims.

### BRIEF DESCRIPTION OF THE DRAWING

FIG. 1 shows an exemplary process that may be used to make combined controlled release bacteria and bacteriophage formulations according to the present invention.

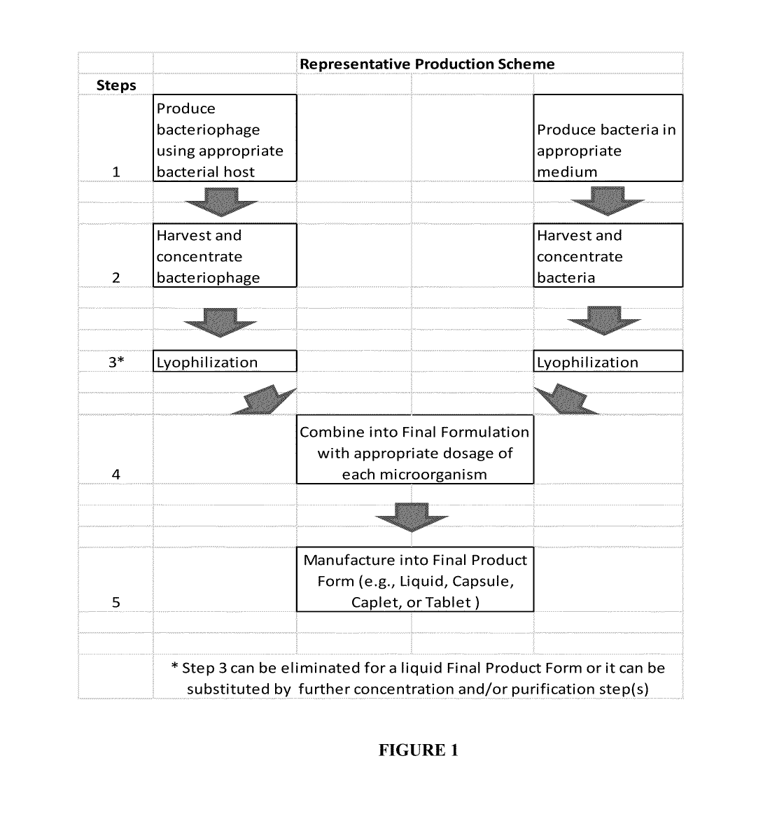

### DETAILED DESCRIPTION

A delivery system is disclosed for the controlled release of one or more BAs into the surrounding environment. Controlled release delivery systems include those systems capable of site specific delivery, extended release, sustained release, delayed release, repeat action, prolonged release, bimodal release, pulsatile release, modified delivery, pH sensitive delivery, and/or target specific delivery, among others. The BAs include, but are not limited to, beneficial microorganisms, such as probiotic bacteria, bacteriophages, DNA, RNA, proteins, components that are bacterial in nature, and modified variations thereof. The solid dosage form may take the form of a tablet, capsule, pill, wafer, or sachet, and is not limited to an orally administered dosage form such as a tablet or capsule.

Probiotic Bacteria Production may generally include the following steps:
1. Bacteria Seed Bank—Individual strains of probiotic bacteria are maintained in a seed bank of pure cultures which are stored as frozen liquid but can also be lyophilized.
2. Inoculation Culture—The production process begins by selecting an appropriate seed culture from the bank to inoculate a relatively small volume of media that is incubated until the desired cell density is reached, this becomes the Inoculation Culture.
3. Production Culture—The Inoculation Culture is used to seed a relatively large volume of media (e.g., 150-450 L) which may serve as an intermediate to a Production Culture (a further scale-up step of 500 L or greater), or it may serve as the final Production Culture. The bacteria in the Production Culture are harvested after sufficient incubation provides a desired cell density.
4. Harvesting—Centrifugation is usually used to harvest the probiotic bacteria from the Production Culture resulting in a slurry, but it could also be harvested by filtration.
5. Lyophilization—The slurry of probiotic bacteria is mixed with cryoprotectants, and then placed into a freeze dryer for lyophilization in order to preserve the bacteria.
6. Storage—The lyophilized bacteria are then stored, preferably under freezing conditions.
7. Formulation—The lyophilized probiotic bacteria may then be combined with excipients, and can also be combined with other probiotic bacteria, for final product preparation into powders, capsules, caplets, tablets, etc., including controlled release formulations.
   Probiotic Bacteriophage Production may generally include the following steps:
1. Bacteriophage Seed Bank—Individual bacteriophages are maintained as pure cultures in a seed bank either in liquid media, or a liquid concentrate or lyophilized. The bacteriophage seed bank is stored as cold as appropriate given the type of culture.
2. Prepare Host Bacteria—The host bacteria are prepared from a seed bank as described above for the production of probiotic bacteria to produce an inoculation, intermediate or production culture of suitable volume for preparation of the desired amount of bacteriophage.
3. Bacteriophage Production—Host bacteria are used to replicate and amplify bacteriophage in a process that can result in several hundred bacteriophage progeny from the infection of a single bacterium by a single parental bacteriophage. A stepwise process is used to scale-up bacteriophage production beginning with a relatively small culture of host bacteria to be infected with a small volume of the seed bacteriophage, followed by serial infection of an intermediate size culture and then a final production size culture to provide the desired quantity of bacteriophage. The amount of bacteriophage added to the host bacteria is usually controlled in order to optimize the multiplicity of infection (m.o.i.) which is the ratio of bacteriophage to bacteria. After sufficient incubation is allowed for replication of the bacteriophage in the host bacteria, the host bacteria can be concentrated just prior to bacteriophage induced lysis or lysis can be completed followed by purification of the bacteriophage from the bacterial debris.
4. Bacteriophage Harvesting—After bacteriophage lysis, bacterial debris can be removed by centrifugation or filtration resulting in a cleared lysate. Bacteriophage can be concentrated from the cleared lysate by centrifugation, precipitation, filtration or a combination thereof. Optionally, the bacteriophage could also be added to a Production Culture of probiotic bacteria for co-harvesting with the probiotic bacteria into the centrifuge slurry.
5. Bacteriophage Lyophilization—Concentrated bacteriophage can be combined with cryoprotectants then placed in a freeze dryer for lyophilization in order to preserve the bacteriophage and transform them into a dry material for formulation. Optionally, the concentrated bacteriophage can also be combined with non-host bacteria, namely probiotic bacteria for co-lyophilization.
6. Formulation—The lyophilized bacteriophage can be combined with excipients and, optionally, probiotic bacteria for formulation into final product forms such as powders, capsules, caplets, tablets, or sachets, etc, including controlled release oral formulations.

Any bacteriophage should be amenable to this general production process. There may be some nuances that would be required for particular bacteriophage, but the overall procedure should be generally applicable. It is expected that although some bacteriophages lose initial titer upon lyophilization, the initial titer number is usually so high that even after a 10-fold loss there is still a substantial population of bacteriophage.

As used herein, a delivery vehicle, for example a homogenously distributed matrix, is made up of hydrophilic agents and/or hydrophobic agents. Hydrophilic agents include swelling, viscosity increasing, gel strength enhancing agents. Hydrophobic agents include waxes and other inert materials, such as ethylcellulose or carnauba wax. More particularly, the hydrophilic agent is selected from at least one of the group, but not limited to: a) a starch selected from the group consisting of corn, rice, or potato starch; b) a hydrophilic gum, polysaccharide, or galactomannan selected from the group consisting of pectin, agar, dextran, carageenan, tragacanth gum, locust beam gum, acacia gum, guar gum, xanthan gum, ghatti gum, alginic acid, or sodium alginate; c) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylhydroxy ethylcellulose, ethylmethylcellulose, hydroxyethylcellulose, cellulose acetate phthalate, or microcrystalline cellulose; d) silica, aluminum silicate, magnesium silicate, aluminum magnesium silicate, sodium silicate or feldspar, e) aluminum hydroxide; f) a protein selected from the group consisting of gelatin or casein; and g) a polymer selected from the group consisting of acrylate, carboxypolymethylene, a polyalkylene glycol, or polyvinylpyrrolidone. In one aspect, the hydrophilic polymers are selected from the group of cellulose derivatives such as microcrystalline cellulose (MCC), hydroxypropyl methylcellulose (HPMC), or hydroxypropyl cellulose (HPC), or from gums and polysaccharides such as guar gum or maltodextrin.

As used herein, optionally, the system may include agents added to aid in gastric bypass or modify the release profile of the one or more BAs due to pH-specific swelling characteristics or site-specific enzyme degradation within the GI tract. These agents may include but are not limited to at least one of alginate, polysaccharides such as gelatin or collagen, guar gum, xanthan gum, pectin, heterogeneous protein mixtures, and polypeptides. The polysaccharides may be pectin and/or an alginate salt, among others. The galactomannan gums may be guar gum, xanthan gum, and/or locust bean gum, among others. The polyethylene derivatives may be polyethylene oxide (PEO) and/or polyethylene glycol (PEG), among others. The hydrolyzed proteins may be gelatin and/or collagen, among others. The polypeptides may be gelatin, collagen, casein, or a heterogeneous protein mixture.

As used herein, the one or more BAs includes, for example, biological agents such as microbes, including probiotic bacteria, bacteriophages, DNA, RNA, protein, modified soil organisms, organisms that compete with lactic acid bacteria, and biopharmaceuticals. The one or more BAs may be viable or non-viable. The one or more BAs may be a beneficial microorganism such as probiotic bacteria or bacteriophages; and in a preferred aspect, the BAs comprise a combination of bacteriophages and probiotic bacteria. The term “probiotic” refers to ingested microorganisms that can live in a host (but may be viable or non-viable upon delivery) and that contribute positively to the host's health and well-being.

As used herein, the electrolytes may be at least one of sodium, potassium, or calcium salts, among others (as used herein, the terms “electrolyte” and “electrolytic agent” are used interchangeably). Through the inclusion of physiologically acceptable electrolytes, the buffered environment allows reconstitution and release to occur under optimal pH conditions for bacterial viability. The interaction between electrolytes and a hydrophilic agent may allow not only the pH-independent release of the one or more BAs, but also allows for the internal pH of the dosage form to remain constant. It is this constant internal pH that contributes significantly to the stability of the biological contents in vivo.

Optionally, physiologically acceptable salts may be introduced to the BA freeze-dried product (FDP) during lyophilization at a ratio of 1.0:0.1 to 1.0:25 FDP to salt. The system ensures the maintenance of a constant pH within the dosage form itself and acts as a cryoprotectant during the freeze-drying process to prevent lysing of the cell.

As used herein, the system may optionally include a desiccant. The desiccant may include, but is not limited to, sodium carboxymethylcellulose, calcium carboxymethylcellulose, colloidal silica dioxide, and combinations thereof. The disintegration agent may include, but is not limited to, croscarmellose sodium sold as Solutab™ available from Blanver Farmoquimica LTDA and crosprovidone (insoluble polyvinylpyrrolidone) sold as Kollidon CL™ available from BASF.

As used herein, the system may optionally include flow and tubing agents. The flow agents may include, but are not limited to, magnesium stearate and stearic acid.

In a first embodiment, the delivery system includes a swelling hydrophilic agent and one or more BAs. It is based on the homologous, or homogeneous, distribution of the various components within a solid matrix dosage form. The system allows for a controlled exposure of the one or more BAs within the dosage form to an aqueous media by controlling the hydration rate of the dosage form via polymer disentanglement and matrix erosion. Optionally, the system may also include a physiologically acceptable electrolyte, a release modifying excipient such as a gum or polysaccharide, a desiccant, and flow or tubing agents, alone or in combination. Electrolytes can provide a mechanism for available water-sequestration to increased stability of the dosage form and the viability of its contents. Desiccants may also be used to sequester available water for a similar purpose. Release modifying excipients, such as gums and polysaccharides, may be used to induce site-specific release through pH-specific swelling or site-specific enzymatic degradation. Flow or tubing agents may be used to improve the manufacturability. This may also result in decreased loss of viability during manufacture due to compression and heat resulting from powder flow, tableting, and encapsulation.

In one aspect of the embodiment, the one or more BAs may be a probiotic bacteria and bacteriophage pre-blend, which can be blended with a carrier. The carrier may be, but is not limited to, monosaccharides or polysaccharides, such as maltodextrin, swellable polymers, such as hydroxypropyl methylcellulose, inert fillers, such as microcrystalline cellulose or di-calcium phosphate, or other inert substances, such as carnauba wax. In the aspect wherein a carrier is included, the carrier may function to assist in the controlled release of the one or more BAs, to aid in the manufacturability of the dosage form, or to increase the stability of the dosage form.

The delivery system can be a readily manufacturable solid dosage form. In one aspect, the dosage form is in the form of a tablet, such as a monolithic tablet, or capsule. When a tablet or capsule, it may be administered orally, anally, and vaginally, among other routes. In one aspect, the dosage form is a monolithic tablet created from a direct-compressible dry blend which does not require processes, such as enteric coating, granulation, or spray drying, that expose the one or more BAs to temperatures that might cause any BAs to be damaged. However, provided such coating or granulation processes are carried out in a manner that do not damage the BAs, nor adversely affect the hydration state of the matrix, they may be amenable.

Release of the one or more BAs into the surrounding environment may be accomplished through a rate-controlled hydration and subsequent swelling of hydrophilic agents. The release of the one or more BAs is determined by the erosion rate and polymeric disentanglement of the swollen hydrophilic matrix. Without subscribing to a particular theory of kinetics, the swelling of the hydrophilic matrix is retarded by a plurality of layers of viscous gelled hydrophilic agents; these gel-states result from the interaction of the hydrophilic agents with the penetrating gastrointestinal fluid. While primarily erosion dependent, the gradual hydration and gelling reaction within the hydrophilic matrix allows for a highly reproducible, programmable release pattern. The programmability of the system allows for nearly any physiologically relevant release pattern to be accomplished. Mathematical treatment of the hydrophilic matrix swelling, erosion, and ensuing release of one or more BAs can be determined, though each model will be representative of the particular components specific to each formulation. This can be accomplished without the need for undue experimentation. Formulation specific to the physical characteristics of each BA, or combinations of BAs, and the desired release profile can be accomplished through both theoretical and empirical means, allowing dissolution of the system and BA or BAs release to occur in a specific physiologic region. Release of contents in a given region of the GI tract is accomplished by the slowly hydrating hydrophilic matrix containing the one or more BAs segregated from the external environment until the desired physiologic region of release, which may be employed to achieve gastric bypass. Consideration of both the area and duration of release is essential in formulation so as to program the system with an appropriate ratio of components to ensure the desired release profile.

The homologous, or homogeneous, distribution of BAs within the hydrophilic matrix provides protection from the fluctuations in pH and exposure to enzymatic degradation present in the external environment. When lyophilized microorganisms are delivered, this isolation from the outside environment allows the microorganisms to remain in lyophilized stasis significantly longer than with conventional immediate release dosage forms.

In another embodiment, when physiologically acceptable electrolytes are included into the delivery system, the electrolyte maintains an intra-dosage form pH irrespective of the external pH. This internal pH may be modified through the selection of electrolytes that are both physiologically-acceptable for human consumption and physiologically-appropriate to the one or more individual BAs. When delivering lyophilized beneficial microorganisms, this internal pH may be selected to create an optimal environment for the reconstitution of the lyophilized microorganisms. Such an environment may result in an increase in viability or activity, or both, during the reconstitution process, and moreover, may limit the exposure of the lyophilized microorganisms to fluctuations in gastrointestinal pH, resulting in an increase in microorganism viability while the matrix is in a hydrated state and prior to the microorganisms' release into the environment.

The addition of physiologically-acceptable electrolytes may also be employed to aid in available water-sequestration. When delivering lyophilized beneficial microorganisms, this is especially useful, as interactions with any available water—such as the available water present in the constituent controlling excipients, flow agents, and desiccants—may result in inadvertent, premature reconstitution prior to release in the gastrointestinal environment. Premature reconstitution from a lyophilized state can cause the microorganisms to begin metabolizing available sources of energy; the constituents of the delivery system provide very limited sources of energy and when these locally available sources of energy are exhausted, the microorganisms expire. The metabolic byproducts of prematurely reanimated organisms may also have a negative impact on the viability of the remaining, non-reanimated organisms. When disposed in a homogeneous manner throughout the dosage form, electrolytic substances that have a higher degree of hydrophilicity than the other constituents of the delivery system surrounding them may preferentially hydrate, decreasing or preventing the re-hydration of the lyophilized agents. An example of a system not including an electrolyte is a system that is dependent upon erosion as its release mechanism, or one in which the maintenance of a constant pH within the dosage form is not desired; lyophilized beneficial microorganisms and hydrophilic agents do not require an electrolyte to make a controlled release dosage form capsule. Another example that does not require an electrolyte is where the controlled release of non-viable beneficial microorganisms (such as non-viable bacterial biomass) is sought as the primary function of the dosage form.

In another embodiment of the delivery system, the addition of release modifying excipients, such as hydrophilic polymers or gums demonstrating pH or enzyme sensitivity, may be employed to alter the swelling or erosion characteristics of the matrix, such as the initiation of swelling or the rate of erosion of the matrix. These release modifying excipients function in combination with the hydrophilic agent to control the release of the one or more BAs. These excipients may be employed to reduce the amount of exposure to the gastric environment by reducing matrix swelling during exposure to gastric pH or during the time the dosage form is expected to transit through the stomach and pylorus. These release modifying excipients may be selected for their in vivo degradation characteristics that occur in localized regions of the gastrointestinal tract. The release modifying agent, when used alone, may function as the hydrophilic agent. One example of this, among many, is that pectin mainly breaks down at the higher pH and enzyme rich environment of the large intestine, thus it can be employed alone as the hydrophilic agent if a greater proportion of lower intestinal tract delivery was desired. Another example among others is that gelatin largely breaks down in the small intestine. With regards to pharmaceutical controlled release formulations, the location of polymer breakdown is of special significance as bioavailability is determined by the amount of drug released within a given timeframe relative to a physiological site of absorption specific to that type of compound. The delivery of one or more BAs is essentially similar in intent, given localized sites for absorption and adsorption. When delivering beneficial microorganisms, the inclusion of release modifying excipients whose swelling characteristics are pH dependent, specifically compounds that preferentially swell in environments above pH 1.0-1.5, is useful for the delivery of lactic acid bacteria that are susceptible to viability losses when exposed to low pH. The low-pH environment will inhibit swelling, thus retarding both beneficial microorganism release and acid-penetration into the dosage form. The inclusion of release modifying excipients whose erosion is enzyme-dependent, specifically compounds that degrade preferentially in the presence of lower intestinal tract enzymes, is useful for the delivery of lactic acid bacteria whose attachment site is distal to the location of the enzymes.

In another embodiment of the delivery system, the system is a pore forming wax matrix composed of one or more inert insoluble waxes, polymers, or fillers in which is disposed pore forming excipients and the active lyophilized bacteria and their associated lyophilized carrier proteins. Hydrophilic agents may be included with hydrophobic agents to make pore forming wax matrices.

In yet another embodiment, the system may include a multi-particulate plurality of granules, coated beads, or coated non-pareils that are distributed within the dosage form in either an active polymer matrix or immediate release matrix for the purposes of controlled release of the lyophilized active ingredients.

In one embodiment, the dosage form disclosed is formed from a pre-blend. When a monolithic tablet, the pre-blend is mixed using dry-blend techniques known to those skilled in the art, and the dosage form is created using a direct compression process. Employing a pre-blend that is formed using dry-blend techniques is a significant improvement over the use of blends resulting from granulation, spheronization-extrusion, or other processes that might expose the biological agents to moisture or solvents and potentially lower the viability of the biological agents. Employing a pre-blend that is capable of forming a monolithic dosage form using only the techniques of direct-compression, in the case of a tablet, or high speed encapsulation, in the case of a capsule, is a significant improvement over manufacturing processes that require multi-stage compression, multiple geometrically-altered components, or coatings that might expose the biological component to hazardous environmental conditions such as solvents, high forces of compression, excessive heat, or undue physical stress. When delivering lyophilized beneficial microorganisms, preventing the premature reconstitution of the organisms is important to maintaining the in vivo viability of the microorganisms.

The dosage form disclosed may be formed from a pre-blend in which a lyophilized biological component, for example a lyophilized beneficial microorganism, is mixed with a pre-blend of one or more controlling excipients, fillers, desiccants, and flow agents that has been mechanically, chemically, or otherwise dried to reduce the available water present for the purpose of preventing undesirable interactions of the beneficial organisms and hydrophilic agents with any available water within the dosage form. The minimization of available water within the dosage form is intended to prevent unintentional or pre-mature reconstitution of the lyophilized organisms. The use of a pre-blend in which the non-lyophilized components are dried and subsequently blended with the lyophilized components, while not necessary for the creation of a controlled release dosage form, is a significant improvement over the use of either non-dried excipients that may contain enough available water to induce pre-mature reconstitution prior to in vivo release, or the drying of a pre-blend containing both lyophilized and non-lyophilized components, which exposes the lyophilized components to undue heat and may extensively reduce their in vivo viability.

Unless otherwise noted, all of the following embodiments are formulated through standard dry blend and direct compression with an appropriate lubricant such as magnesium stearate or stearic acid.

In the first embodiment, a formulation is disclosed combining the one or more BAs lyophilized (freeze-dried) powder pre-blend (“FDP”) with a suitable hydrophilic agent such as HPMC, MCC, or PEO, in a ratio of about 1.0:0.1 to 1:25 FDP to hydrophilic agent.

In the second embodiment, a formulation is disclosed including the one or more BAs FDP, hydrophilic agent, and a physiologically acceptable electrolyte such as NaHCO₃, Na₂ CO₃, or Ca CO₃, in a ratio of about 1.0:0.1:0.1 to 1:25:25 FDP to hydrophilic agent to electrolyte.

In the third embodiment, a formulation is disclosed including the one or more BAs FDP, a hydrophilic agent, and a release modifying agent in the form of a hydrophilic polysaccharide such as pectin or sodium alginate alginic acid, or a gum such as xanthan gum, guar gum, locust bean gum, or tragacanth gum, in a ratio of about 1.0:0.1:0.1 to 1:25:25 FDP to hydrophilic agent to polysaccharide or gum.

In the fourth embodiment, a formulation is disclosed including the one or more BAs FDP, a hydrophilic agent, a release modifying agent in the form of a hydrophilic polysaccharide or gum, and a physiologically acceptable salt in a ratio of about 1.0:0.1:0.1:0.1 to 1:25:25:25 FDP to hydrophilic agent to polysaccharide or gum to electrolyte.

In the fifth embodiment, a formulation is disclosed including the one or more BAs FDP, a hydrophilic agent, a release modifying agent in the form of a hydrophilic polysaccharide or gum, a physiologically acceptable salt, and an inert filler in a ratio of about 1.0:0.1:0.1:0.1:0.1 to 1:25:25:25:25 FDP to hydrophilic agent to polysaccharide or gum to electrolyte to inert filler.

In the sixth embodiment, a formulation is disclosed combining the lyophilized lactic acid bacteria and bacteriophage pre-blend with a suitable hydrophobic agent such as carnauba wax, in a ratio of about 1.0:0.1 to 1:25 FDP to hydrophobic agent.

In the seventh embodiment, a formulation is disclosed including the one or more BAs FDP, a hydrophobic agent, and a physiologically acceptable electrolyte such as NaHCO₃, Na₂ CO₃, or Ca CO₃, in a ratio of about 1.0:0.1:0.1 to 1:25:25 FDP to hydrophobic agent to electrolyte.

In the eighth embodiment, a formulation is disclosed including the one or more BAs FDP, a hydrophobic agent, a physiologically acceptable electrolyte such as NaHCO₃, Na₂ CO₃, or Ca CO₃, and a release modifying agent in the form of a hydrophilic polysaccharide such as pectin or sodium alginate alginic acid, or a gum such as xanthan gum, guar gum, locust bean gum, or tragacanth gum, in a ratio of about 1.0:0.1:0.1:0.1 to 1:25:25:25 FDP to hydrophobic agent to polysaccharide or gum to electrolyte.

The dosage forms may be, for example, monolithic tablets or gelatin or vegetable capsules or sachets for oral, anal, or vaginal delivery.

### Methods

The formulations described herein can be prepared in accordance with the following methods. In these formulations, tablets can be prepared using a method of dry blending and direct compression using a Carver hydraulic press or a rotary tablet press. Evaluations can be performed using a USP Type II (paddle) dissolution apparatus.

Dosage forms according to the present invention may be tested by exposing the dosages to 1000 mL 0.1N HCl for 2 hours at 50 RPM. The dosages can then be removed and placed into peptone buffer medium, such as KH₂PO₄ or peptone buffer dissolution medium, and stomached, (the dosage form is crushed and homogenized within the buffer media for the purpose of enumerating the remaining bacteria in the tablet), after which a sample can be taken from the dissolution media. The samples are then plated on MRS and RCM media to discern viable colony forming units (CFU), or filtered, reacted with 4′,6-diamidino-2-phenylindole, and enumerated under UV-light. The bacteriophage can be plated with an indicator host bacterium on appropriate media resulting in a lawn of host bacteria that reveals visible plaque forming units (PFU) of viable bacteriophage.

Dosage form stability can be tested, for example, by packaging the dosage formed in foil sachets which are then exposed to ambient environmental conditions (25 degrees C., 60% Relative Humidity) for 4 months and subsequently tested. These samples can then be removed to peptone buffer solution, stomached, and plated on MRS and RCM media to discern viable colony forming units (CFU). The bacteriophage can be plated with an indicator host bacterium on appropriate media resulting in a lawn of host bacteria that reveals visible plaque forming units (PFU) of viable bacteriophage.

### Example 1

A monolithic tablet of approximately 382 mg having a hydrophilic agent and a combination of BAs, including probiotic bacteria and bacteriophages can be prepared as shown in Table 1. Here, the beneficial microorganisms include one or more lactic acid bacteria and bacteriophage pre-blends of lyophilized powder and starch, and the hydrophilic agent employed is microcrystalline cellulose (MCC), maltodextrin, hydroxypropyl methylcellulose (HPMC), or polyethylene oxide (PEO). The included bacteriophages can be selected based on their specificity for detrimental target bacteria. The addition of the hydrophilic agent will retard the release of the BAs from the dosage form. Stearic acid is included as a flow agent and silica is employed as a flow agent and desiccant.

It is expected that testing of this formulation will reflect a level of controlled release granted through the use of a matrix comprised of a hydrophilic agent and the BAs. This controlled release can be shown by a much higher level of viable lactic acid bacteria colony forming units (CFU) and bacteriophages (PFU) delivered after exposure to gastric media than the control. The use of less swellable hydrophilic agents such as MCC and maltodextrin may be associated with sufficient, but lower levels of control. A superior level of control may be demonstrated in both polyethylene oxide and HPMC matrices. Thus, the hydrophilic agent is not limited to a particular type of hydrophilic agent, so long as sufficient matrix viscosity is achieved.

TABLE 1

| | A1 | | | | |
|---|---|---|---|---|---|
| Dosage Formulas (mg) | (CTRL) | A2 | A3 | A4 | A5 |
| Pre-blend(s): Lactic acid bacteria | 150 | 150 | 150 | 150 | 150 |
| and bacteriophages | | | | | |
| HPMC | 0 | 0 | 0 | 200 | 0 |
| PEO | 0 | 0 | 0 | 0 | 200 |
| MCC | 0 | 200 | 0 | 0 | 0 |
| Maltodextrin | 0 | 0 | 200 | 0 | 0 |
| Stearic Acid | 16 | 16 | 16 | 16 | 16 |
| Silica | 16 | 16 | 16 | 16 | 16 |
| TOTAL WEIGHT | 182 | 382 | 382 | 382 | 382 |

### Example 2

A monolithic tablet of approximately 382 mg containing a hydrophilic agent, an electrolytic agent, and one or more BAs can be prepared as set forth in Table 2, with B1 as the control group. The formulation employs HPMC as the hydrophilic agent, NaHCO₃, Na₂CO₃, or NaH₂PO₄ as the electrolytic agent, and one or more lactic acid bacteria and bacteriophage pre-blends of lyophilized powder and starch as the one or more BAs. The addition of NaHCO₃, Na₂CO₃, or NaH₂PO₄ is expected to establish the pH within the dosage form. Stearic acid can be included as a flow agent and silica can be employed as a flow agent and desiccant.

It is expected that the internal pH of the dosage form can be altered by the presence of an electrolyte, affecting the amount of active viable CFU and bacteriophages PFU delivered. This establishment of a particular internal pH is associated with differing levels of viability for a given reconstituted lyophilized organism. In particular, formulation B2, which contains Na₂CO₃, may provide an internal pH which aides in the reconstitution of viable lactic acid bacteria and bacteriophages.

TABLE 2

| Dosage Formulas (mg) | B1 (ctrl) | B2 | B3 | B4 |
|---|---|---|---|---|
| Pre-blend(s): Lactic acid bacteria | 150 | 150 | 150 | 150 |
| and bacteriophages | | | | |
| HPMC | 00 | 100 | 100 | 100 |
| NaHCO₃ | 0 | 100 | 0 | 0 |
| NaHCO₃ | 0 | 0 | 100 | 0 |
| NaH₂PO₄ | 0 | 0 | 0 | 100 |
| Stearic Acid | 16 | 16 | 16 | 16 |
| Silica | 16 | 16 | 16 | 16 |
| TOTAL WEIGHT | 182 | 382 | 382 | 382 |

### Example 3

A monolithic tablet of approximately 382 mg containing a hydrophilic agent, a release-modifying excipient, and one or more BAs can be prepared as shown in Table 3, with C1 as the control group. The formulation employs HPMC as the hydrophilic agent, pectin or gelatin as the release-modifying excipient, and one or more lactic acid bacteria and bacteriophage pre-blends of lyophilized powder and starch as the one or more BAs. Stearic acid can be included as a flow agent and silica can be employed as a flow agent and desiccant.

It is expected that an increased level of control is possible when release modifying excipients are added to a hydrophilic swellable matrix. The presence of pectin or gelatin is associated with a degree of pH-dependent degradation and an overall increase in matrix viscosity which retards the release of the one or more BAs. This can be reflected in the increase in viable CFU and bacteriophages PFU delivered after exposure to gastric pH.

TABLE 3

| Dosage Formulas (mg) | C1 (CTRL) | C2 | C3 |
|---|---|---|---|
| Pre-blend(s): Lactic acid bacteria | 150 | 150 | 150 |
| and bacteriophages | | | |
| HPMC | 0 | 100 | 100 |
| Pectin | 0 | 100 | 0 |
| Gelatin | 0 | 0 | 100 |
| Stearic Acid | 16 | 16 | 16 |
| Silica | 16 | 16 | 16 |
| TOTAL WEIGHT | 182 | 382 | 382 |

### Example 4

A monolithic tablet of approximately 382 mg containing a hydrophilic agent and one or more BAs can be prepared as shown in Table 4 with C1 as the control group. The formulation employs pectin as the hydrophilic agent and one or more lactic acid bacteria and bacteriophage pre-blends of lyophilized powder and starch as the one or more BAs. Stearic acid can be included as a flow agent and silica can be employed as a flow agent and desiccant.

An increased level of control can be possible when employing a hydrophilic agent that displays pH-dependent and enzyme-dependent degradation. A release-modifying agent may be used as a hydrophilic agent. The presence of pectin may also be associated with an overall increase in matrix viscosity which retards the release of the BAs. This can be reflected in the increase in viable CFU and bacteriophages PFU delivered after exposure to gastric pH.

TABLE 4

| Dosage Formulas (mg) | C1 (CTRL) | C4 |
|---|---|---|
| Pre-blend(s): Lactic acid bacteria | 150 | 150 |
| and bacteriophages | | |
| Pectin | 0 | 200 |
| Stearic Acid | 16 | 16 |
| Silica | 16 | 16 |
| TOTAL WEIGHT | 182 | 382 |

### Example 5

A monolithic tablet of approximately 482 mg containing a hydrophilic agent, a release-modifying excipient, an electrolytic agent, and one or more lactic acid bacteria and bacteriophage pre-blends of lyophilized powder and starch as the one or more BAs can be prepared as shown in Table 5 with D1 as the control group. The formulation employs guar gum as the hydrophilic agent, pectin as the release-modifying excipient, NaHCO₃ as the electrolytic agent, and one or more lactic acid bacteria and bacteriophage pre-blends of lyophilized powder and starch as the one or more BAs. Stearic acid can be included as a flow agent and silica can be employed as a flow agent and desiccant.

Here, galactomannan gum can be used as a hydrophilic agent in combination with a sodium salt and a polysaccharide in a hydrophilic swellable matrix. The presence of a galactomannan gum is expected to be associated with an overall increase in matrix viscosity which retards the release of the BAs, and the presence of NaHCO₃ is expected to be associated with internal pH modulation favorable to the reconstitution of lactic acid bacteria. This can be reflected in the increase in viable CFU and bacteriophages PFU delivered after exposure to gastric pH.

TABLE 5

| Dosage Formulas (mg) | D1 (CTRL) | D2 |
|---|---|---|
| Pre-blend(s): Lactic acid bacteria | 150 | 150 |
| and bacteriophages | | |
| Guar | 0 | 100 |
| NaHCO₃ | 0 | 100 |
| Pectin | 0 | 100 |
| Stearic Acid | 16 | 16 |
| Silica | 16 | 16 |
| TOTAL WEIGHT | 182 | 482 |

### Example 6

A monolithic tablet of approximately 534 mg containing a hydrophilic agent, an electrolytic agent, a release-modifying excipient, a filler, and one or more BAs can be prepared as shown in Table 6. The formulation employ HPMC as the hydrophilic polymer, NaHCO₃ as the electrolytic agent, pectin as the release-modifying excipient, MCC as the filler, and one or more lactic acid bacteria and bacteriophage pre-blends of lyophilized powder and starch as the one or more BAs. It is expected that the addition of inert filler is associated with increased powder flowability, which is often advantageous during manufacture. Stearic acid is included as a flow agent and silica is employed as a flow agent and desiccant. Turmeric is included as a colorant.

It is expected that this formulation will provide controlled release of viable BAs over an extended duration. It is further expected that the controlled release of the hydrophilic matrix will perform similarly regardless of the duration of exposure to gastric media.

TABLE 6

| Dosage Formulas (mg) | E1 | E2 |
|---|---|---|
| Pre-blend(s): Lactic acid bacteria | 150 | 150 |
| and bacteriophages | | |
| HPMC | 50 | 50 |
| NaHCO₃ | 50 | 50 |
| MCC | 200 | 200 |
| Pectin | 50 | 50 |
| Stearic Acid | 16 | 16 |
| Silica | 16 | 16 |
| Turmeric | 2 | 2 |
| TOTAL WEIGHT | 534 | 534 |

### Example 7

A monolithic tablet of approximately 443 mg containing a hydrophilic agent, an electrolytic agent, a release-modifying excipient, a filler, and one or more BAs can be prepared as shown in Table 6. It is expected that such formulations can provide controlled release of bacteria and bacteriophage over an extended duration, for example, from zero to eight hours, and that the rate of release will be linear from zero until approximately eight hours.

### Example 8

A monolithic tablet of approximately 532 mg containing a hydrophilic agent, an electrolytic agent, a release-modifying excipient, a filler, and one or more BAs can be prepared as shown in Table 8. The hydrophilic agent employed is HPMC or PEO, the electrolytic agent is NaHCO₃, the release-modifying excipient employed is pectin, the filler employed is MCC and one or more bifidobacterium and bacteriophage pre-blends of lyophilized powder and starch as the one or more BAs. Stearic acid is included as a flow agent and silica is employed as a flow agent and desiccant. Turmeric is included as a colorant.

It is expected that such formulations can provide controlled release of bacteria and bacteriophage over an extended duration, and that the hydrophilic matrix can release the bifidobacterium in a favorable profile, i.e., after eight hours. Such formulations may be useful to delivering the bacteria and bacteriophage to the lower intestine and beyond.

TABLE 8

| Dosage Formulas (mg) | F2 | F3 |
|---|---|---|
| Pre-blend(s): Bifidobacterium acid | 150 | 150 |
| bacteria and bacteriophages | | |
| HPMC | 150 | 0 |
| PEO | 0 | 150 |
| Pectin | 100 | 100 |
| NaHCO₃ | 100 | 100 |
| Stearic Acid | 16 | 16 |
| Silica | 16 | 16 |
| TOTAL WEIGHT | 532 | 532 |

### Example 9

Two-piece capsules of approximately 665 mg containing two hydrophilic agents, an electrolytic agent, a release-modifying excipient, and one or more BAs can be prepared as shown in Table 9 with G1 as the control group. The hydrophilic agents employed are HPMC and Guar, the electrolytic agent is NaHCO₃, the release-modifying excipient employed is pectin and the one or more lactic acid bacteria and bacteriophage pre-blends of lyophilized powder and starch are the one or more BAs. Stearic acid is included as a flow agent and silica is employed as a flow agent and desiccant.

It is expected that the combination of a hydrophilic agents, an electrolyte, and a release-modifying excipient are capable of controlling the release of the one or more BAs from a capsule. Dosage form flexibility, such as formulation for a tablet or capsule, provides substantial adaptability during manufacture.

TABLE 9

| Dosage Formulas (mg) | G1 (CTRL) | G2 |
|---|---|---|
| Pre-blend(s): Lactic acid bacteria | 150 | 150 |
| and bacteriophages | | |
| Pectin | 0 | 75 |
| HPMC | 0 | 110 |
| NaHCO₃ | 0 | 110 |
| Guar | 0 | 200 |
| Stearic Acid | 10 | 10 |
| Silica | 10 | 10 |
| TOTAL WEIGHT | 170 | 665 |

### Example 10

Monolithic tablets of approximately 684 mg and 342 mg containing a hydrophilic agent, an electrolytic agent, a release-modifying excipient, a filler, and one or more BAs can be prepared as shown in Table 10. The hydrophilic polymer employed is HPMC, the electrolytic agent is NaHCO₃, the release-modifying excipient employed is pectin, the filler employed is MCC, and the one or more lactic acid bacteria and bacteriophage pre-blends of lyophilized powder and starch are the one or more BAs. Stearic acid is included as a flow agent and silica is employed as a flow agent and desiccant.

It is expected that the combination of a hydrophilic agent, electrolyte, and a release-modifying excipient are capable of geometric scalability, tablet shape, size, and volume variation while controlling the release of the one or more BAs from the matrix. This flexibility is especially useful in manufacture when differing formulation volumes are required when altering tablet shapes and sizes.

TABLE 10

| Dosage Formulas (mg) | H1 | H2 |
|---|---|---|
| Pre-blend(s): Lactic acid bacteria | 75 | 150 |
| and bacteriophages | | |
| Pectin | 50 | 100 |
| HPMC | 50 | 100 |
| NaHCO₃ | 50 | 100 |
| Guar | 100 | 200 |
| Stearic Acid | 8 | 16 |
| Silica | 8 | 16 |
| Turmeric | 1 | 2 |
| TOTAL WEIGHT | 342 | 684 |

### Example 11

Monolithic tablets of approximately 668 mg containing a hydrophilic agent, an electrolytic agent, a release-modifying excipient, a filler, and one or more BAs can be prepared as shown in Table 11. The hydrophilic polymer employed is HPMC, the electrolytic agent is NaHCO₃, the release-modifying excipient employed is pectin, the filler employed is MCC, and the one or more lactic acid bacteria and bacteriophage pre-blends of lyophilized powder and starch are the one or more BAs. Stearic acid is included as a flow agent and silica is employed as a flow agent and desiccant. Turmeric is included as a colorant.

It is expected that the beneficial effects of drying excipients before tableting will be evidenced by the increase in viable lactic acid bacteria CFU and bacteriophage PFU present in the dried pre-blend.

TABLE 11

| Dosage Formulas (mg) | I1 | I2 |
|---|---|---|
| Pre-blend(s): Lactic acid bacteria | 150 | 150 |
| and bacteriophages | | |
| HPMC | 100 | 100 |
| Pectin | 100 | 100 |
| NAH(CO3)2 | 100 | 100 |
| MCC | 200 | 200 |
| Stearic Acid | 8 | 8 |
| Silica | 8 | 8 |
| Turmeric | 2 | 2 |
| TOTAL WEIGHT | 668 | 668 |

### Example 12

A monolithic tablet of approximately 342 mg containing a hydrophilic agent, an electrolytic agent, a release-modifying excipient, a filler, and one or more BAs can be prepared as shown in Table 12. The hydrophilic agent employed is HPMC of viscosity 4000 mPa or 15000 mPa, the electrolytic agent is NaHCO₃, the release-modifying excipient employed is pectin, the filler employed is MCC and the one or more bifidobacterium and bacteriophage pre-blends of lyophilized powder and starch are the one or more BAs. Stearic acid is included as a flow agent and silica is employed as a flow agent and desiccant. Turmeric is included as a colorant.

It is expected that such formulations will demonstrate the capacity for differential controlled release of viable BAs by employing hydrophilic agents of differing viscosities.

TABLE 12

| Dosage Formulas (mg) | H1 | H2 |
|---|---|---|
| Pre-blend(s): Bifidobacterium acid | 75 | 75 |
| bacteria and bacteriophages | | |
| HPMC, 4000 mPa | 50 | 0 |
| HPMC, 15000 mPa | 0 | 50 |
| Pectin | 50 | 50 |
| NaHCO₃ | 50 | 50 |
| MCC | 100 | 100 |
| Stearic Acid | 8 | 8 |
| Silica | 8 | 8 |
| Turmeric | 1 | 1 |
| TOTAL WEIGHT | 342 | 342 |

### Example 13

A monolithic tablet of approximately 343 mg containing a hydrophilic agent, an electrolytic agent, a release-modifying excipient, a filler, and one or more BAs can be prepared as shown in Table 13. The hydrophilic agent employed is HPMC, the electrolytic agent is NaHCO₃, the release-modifying excipient employed is pectin, the filler employed is MCC and the one or more lactic acid bacteria and bacteriophage pre-blends of lyophilized powder and starch are the one or more BAs. Stearic acid is included as a flow agent and silica is employed as a flow agent and desiccant. Turmeric is included as a colorant.

It is expected that these formulations will demonstrate the capacity for increased stability over time when stored in an ambient environment, (25 degrees C., 60% Relative Humidity), evidenced by a relatively constant amount of viable lactic acid bacteria CFU and bacteriophage PFU.

TABLE 13

| Dosage Formulas (mg) | K1 |
|---|---|
| Pre-blend(s): Lactic acid bacteria and | 75 |
| bacteriophages | |
| HPMC | 50 |
| Pectin | 50 |
| NaHCO₃ | 50 |
| MCC | 100 |
| Stearic Acid | 8 |
| Silica | 8 |
| Turmeric | 2 |
| TOTAL WEIGHT | 343 |

### Kits and Marketing

The present invention also includes kits comprising one or more controlled release formulations that include both probiotic bacteria and bacteriophages together with labeling and, optionally including printed instructions indicating the use of the formulations to effect changes in a resident microorganism population in a human or animal gastrointestinal tract.

The present invention also includes methods of marketing controlled release dosage forms comprising probiotic microbes and bacteriophages. Such marketing may include, for example, labeling or other written or verbal indications regarding the beneficial use of the dosage forms. For example, such marketing may indicate that a mammalian subject may improve their health or effect a change in their resident gastrointestinal tract microorganism populations by using the dosage forms of the present invention. Further, the marketing may promote the beneficial additive and subtractive nature of the combined probiotic bacteria and bacteriophage controlled release dosage forms of the present invention.

The discussion above is descriptive, illustrative and exemplary and is not to be taken as limiting the scope defined by any appended claims.

We claim:

1. A controlled release dosage form comprising probiotic microbes and bacteriophages, wherein the dosage form provides for simultaneous release of probiotic bacteria and bacteriophages.

2. The controlled release dosage form of claim 1, wherein the probiotic bacteria and bacteriophages are provided as parts of a homogeneous mixture.

3. The controlled release dosage form of claim 1, wherein the dosage form is a tablet.

4. The controlled release dosage form of claim 3, wherein the dosage form is a tablet without an enteric coating.

5. The controlled release dosage form of claim 1, further comprising a hydrophilic agent, a release modifying agent, and an electrolytic agent.

6. The controlled release dosage form of claim 1, wherein the dosage form is characterized by the inclusion of additive probiotic bacteria and subtractive bacteriophages.

7. The controlled release dosage form of claim 1, wherein the probiotic bacteria and bacteriophages are released in the upper portion of a human or animal gastrointestinal tract.

8. The controlled release dosage form of claim 1, wherein the probiotic bacteria and bacteriophages are released in the lower portion of a human or animal gastrointestinal tract.

9. A method for making a controlled release probiotic bacteria and bacteriophage dosage form, comprising:
selecting probiotic bacteria;
selecting bacteriophages;
combining the probiotic microbes and bacteriophages together; and
forming a pre-blend comprising both the probiotic bacteria and the bacteriophages.

10. The method of claim 9, further comprising:
identifying target bacteria, wherein the target bacteria are one of pathogenic bacteria and non-pathogenic bacteria; and
selecting one or more bacteriophages for inclusion in the oral dosage form based on the identified target bacteria.

11. The method of claim 9, further comprising compressing the pre-blend into a dosage form.

12. The method of claim 11, wherein the dosage form is a tablet.

13. The method of claim 12, wherein the tablet is without an enteric coating.

14. The method of claim 9, further comprising including a hydrophilic agent, a release modifying agent, and an electrolytic agent in the homogenous pre-blend.

15. The method of claim 9, wherein the bacteriophages are lyophilized.

16. A method of treating a human or animal gastrointestinal tract comprising: providing a controlled oral release dosage form comprising probiotic bacteria and bacteriophages to a human or animal subject.

17. The method of claim 16, wherein the bacteriophages are included in the dosage form to subtract unwanted bacteria populations in the human or animal gastrointestinal tract.

18. The method of claim 17, wherein the included bacteriophages are specific to one or more pathogenic bacteria hosts.

19. The method of claim 17, wherein the included bacteriophages are specific to one or more non-pathogenic bacteria hosts.

20. The method of claim 16, wherein the controlled oral release dosage form is a tablet.
